(19) United States
(12) Patent Application Publication
(10) Pub. No.: US 2025/0302518 A1
(43) Pub. Date: Oct. 2, 2025

(54) POLYHEDRAL FIDUCIAL MARKERS FOR COMPUTER-ASSISTED SURGERY

(71) Applicants: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(72) Inventors: Nathan ZAMARRIPA, Kittery Point, ME (US); Rui Jorge Melo TEIXEIRA, Coimbra (PT); Paul Alexander TORRIE, Marblehead, MA (US); Rafal Z. JEZIERSKI, Candia, NH (US); Philip A. CORMIER, Newburyport, MA (US); Luis Carlos Fial Teixeira RIBEIRO, Coimbra (PT); Joao Pedro de Almeida BARRETO, Coimbra (PT)

(73) Assignees: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(21) Appl. No.: 18/873,168

(22) PCT Filed: Jun. 8, 2023
(86) PCT No.: PCT/US2023/024833

§ 371 Date: Dec. 9, 2024

Related U.S. Application Data
(60) Provisional application No. 63/393,063, filed on Jul. 28, 2022.

(51) Int. Cl. A61B 17/88; A61B 17/00; A61B 17/86; A61B 34/20; A61B 90/00

(52) U.S. Cl. CPC A61B 17/8891; A61B 17/862; A61B 17/866; A61B 90/39; A61B 2017/00876; A61B 2034/2065; A61B 2090/363; A61B 2090/3916

(57) ABSTRACT

Disclosed are systems and methods for polyhedral fiducial marker configurations that improve the mechanisms for their placement as well how they can be relied upon for surgery. The disclosed fiducial markers are configured with polyhedral shapes and increased marker surfaces, and improved marker designs that offer improved installation and system tracking, which leads to improved procedural efficiency and outcomes from surgery. The disclosed polyhedral fiducial markers can have a shape geometry with a number of faces that is an increase from existing markers. This provides improved mechanisms for installation and tracking with a camera system. Moreover, improved installation tools for the polyhedral fiducial markers can be utilized, which can be based on how the fiducial marker is held at the distal end of the installation tool. Such categorical installation tools can include: magnet-based embodiments; clip-based embodiments (e.g., O-ring and spring clip variants); and friction-based embodiments.

### CROSS-REFERENCE TO RELATED APPLICATIONS

This application claims the benefit of U.S. Provisional Application No. 63/393,063 filed 28 Jul. 2022 and titled “Polyhedral Fiducial Markers For Computer-Assisted Surgery.” The provisional application is incorporated by reference herein as if reproduced in full below.

### TECHNICAL FIELD

The present disclosure generally relates to computer-assisted surgery, and more particularly, to polyhedral fiducial marker installation tools, and the usage of polyhedral fiducial markers for computer-assisted surgery.

### BACKGROUND

Conventional soft tissue and ligament surgeries, for example, an anterior cruciate ligament (ACL) reconstruction procedure, utilize navigational technologies to aid in their performance. For example, video-based technologies can be utilized to not only perform the surgery, but also to place the fiducial markers that are relied upon during the surgery.

### SUMMARY

The disclosed systems and methods provide novel fiducial marker configurations that improve the mechanisms for their placement, in addition to how they can be relied upon for successful surgery. As discussed in more detail below, the disclosed systems and methods provide polyhedral fiducial markers configured with polyhedral shapes and increased marker surfaces, and improved marker designs that offer improved installation and system tracking, which leads to improved procedural efficiency and outcomes from surgery.

By way of background, navigational-based surgical technologies can use video-based navigation to assist surgeons in accurately placing femoral tunnels during ACL reconstruction. Critical to the procedure is the placement of the fiducial marker(s). According to some embodiments, the marker(s) can contain unique patterns that the arthroscope can detect and view, and serves as a datum enabling video-based navigation.

However, conventional marker shapes, patterns, and configurations can cause losses in tracking connections with the camera systems during a procedure, resulting in intermittent connectivity that can not only slow down the overall speed and efficiency of the procedure, but also can lead to errors or mistakes (which can ultimately lead to the need for a revision procedure). For example, an existing fiducial marker's face (e.g., based on a cube shape), which is typically parallel to the arthroscope lens, can have a female thread to secure the marker to the driver when placing and removing the marker. However, the female thread itself can create a space of unusable surface area on the marker thereby leading to the intermittent connectivity discussed above.

Therefore, as discussed herein, the disclosed systems and methods provide novel configurations of polyhedral fiducial markers and mechanisms for installation of such markers.

According to some embodiments, the disclosed polyhedral fiducial markers can include a number of faces that is an increase in the number of faces from existing markers (e.g., increased surface area(s) for placement of fiducial markers). This, as evidenced from the discussion herein, provides improved mechanisms for installation and tracking with a camera system. Indeed, the increased number of faces can have a proportional increase in a number of patterns that can be used (e.g., a pattern per face). In some embodiments, each face can have a same or alternative (or different) pattern that is trackable and detectable by a camera system.

As discussed below, the disclosed polyhedral fiducial markers can have at least a predetermined number of more faces that existing markers (e.g., four or more). In some embodiments, rather than simply utilizing a cube for a marker (e.g., which conventionally has four usable faces for fiducial markings), as in conventional marker systems, the disclosed systems and methods can utilize additional shapes, thereby not only increasing the surface area of the marker, but also the number of faces for which a camera can connect to and rely upon during a procedure. For example, a disclosed polyhedral fiducial marker can include an evolved geometry from a cube to a hexahedron, as discussed in more detail below.

Moreover, according to some embodiments, the disclosed systems and methods can additionally utilize improved installation tools for the polyhedral fiducial markers. As discussed in more detail below, the installation tools can be organized into at least three categories based on how the fiducial marker is held at the distal end of the installation tool. According to some embodiments, as discussed in more detail below, such categorical installation tools can include: 1) magnet-based embodiments; 2) clip-based embodiments (e.g., O-ring and spring clip variants); and 3) friction-based embodiments.

According to some embodiments, a method is disclosed for a polyhedral fiducial marker, and its installation and usage during a surgical procedure. According to some embodiments, the method can correspond to the installation of a fiducial marker (also referred to as a bone marker, used interchangeably). In some embodiments, the method can involve retaining at least the bone marker at the distal end of an installation tool, where the installation tool defines a longitudinal central axis and a handle on the proximal end. The method can continue via installing the bone marker into a bone by turning the installation tool about the longitudinal central axis, and then disconnecting the installation tool from the bone marker and leaving the bone marker in the bone by pulling the installation tool away from the bone without interacting with a mechanical release on the handle.

According to some embodiments, an installation tool for a bone marker is disclosed. In some embodiments, the installation tool can include an elongated shaft defining a distal end and a proximal end and a handle disposed at the proximal end of the elongated shaft. The installation tool can further include a receptacle disposed at the distal end, where the receptacle defines an inside surface having a shape complementary to an outside surface of a bone marker. The installation tool can further include a means for retaining and releasing the bone marker from the receptacle, where the means for retaining and release is operable without interaction by a user interacting with the handle.

In some embodiments, the means for retaining and releasing can further include a magnet disposed within the elongated shaft proximal to the receptacle. In some embodiments, the means for retaining and releasing can further include a retention member disposed in operational relationship to the receptacle, where the retention member is configured to interact with a notch or groove of the bone marker to retain bone marker within the receptacle. In some embodiments, the retention member can be at least one selected from a group consisting of: an elastomeric material; an O-ring; and a spring.

In some embodiments, the means for retaining and releasing can further include a friction member configured to be disposed between the bone marker and the inside surface of the receptacle. In some embodiments, the friction member can include an inside surface configured to abut an outside surface of the bone marker, and an outside surface defining a protrusion, where the protrusion can be configured to interact with an aperture through a side wall of the receptacle. In some embodiments, the friction member holds the bone marker within the receptacle by friction force, and the installation tool is configured to release the bone marker by a force applied along the longitudinal central axis overcoming the friction force.

### BRIEF DESCRIPTION OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIGS. 1A-1B illustrate non-limiting example embodiments of a polyhedral fiducial marker configuration according to some embodiments of the present disclosure;

FIG. 2 illustrates a non-limiting example embodiment of a polyhedral fiducial marker configuration according to some embodiments of the present disclosure;

FIGS. 3A-3B illustrate non-limiting example embodiments of a polyhedral fiducial marker configuration according to some embodiments of the present disclosure;

FIG. 4 illustrates a non-limiting example embodiment of a polyhedral fiducial marker according to some embodiments of the present disclosure;

FIGS. 5A-5B illustrate non-limiting example embodiments of a polyhedral fiducial marker according to some embodiments of the present disclosure; and

FIGS. 6A-6C illustrate non-limiting example embodiments of a polyhedral fiducial marker configuration according to some embodiments of the present disclosure.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

Unless limited otherwise, the terms “connected,” “coupled,” and “mounted,” and variations thereof herein are used broadly and encompass direct and indirect connections, couplings, and mountings. In addition, the terms “connected” and “coupled,” and variations thereof are not restricted to physical or mechanical connections or couplings. Further, terms such as “up,” “down,” “bottom,” “top,” “front,” “rear,” “upper,” “lower,” “upwardly,” “downwardly,” and other orientational descriptors are intended to facilitate the description of the exemplary embodiments of the present disclosure, and are not intended to limit the structure of the exemplary embodiments of the present disclosure to any particular position or orientation. Terms of degree, such as “substantially” or “approximately,” are understood by those skilled in the art to refer to reasonable ranges around and including the given value and ranges outside the given value, for example, general tolerances associated with manufacturing, assembly, and use of the embodiments. The term “substantially,” when referring to a structure or characteristic, includes the characteristic that is mostly or entirely present in the characteristic or structure.

The principles discussed herein provide novel and improved fiducial (or bone) marker configurations and installation mechanisms. According to some embodiments, as discussed below, the disclosed fiducial markers are configured with polyhedral shapes and increased marker surfaces, and improved marker designs that offer improved installation and system tracking, which leads to improved procedural efficiency and outcomes from surgery. The disclosed polyhedral fiducial markers can have a shape geometry with a number of faces that is an increase from existing markers. This provides improved mechanisms for installation and tracking with a camera system. Moreover, improved installation tools for the polyhedral fiducial markers can be utilized, which can be based on how the fiducial marker is held at the distal end of the installation tool.

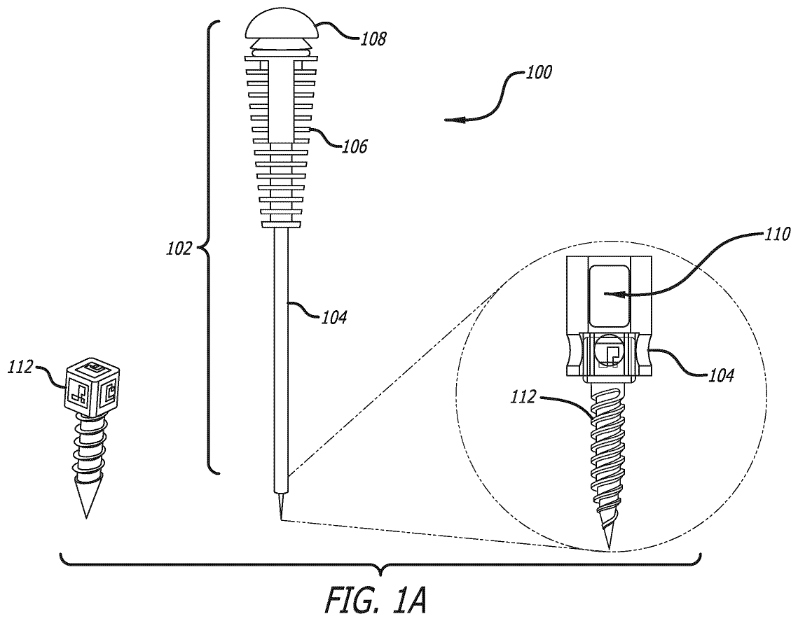

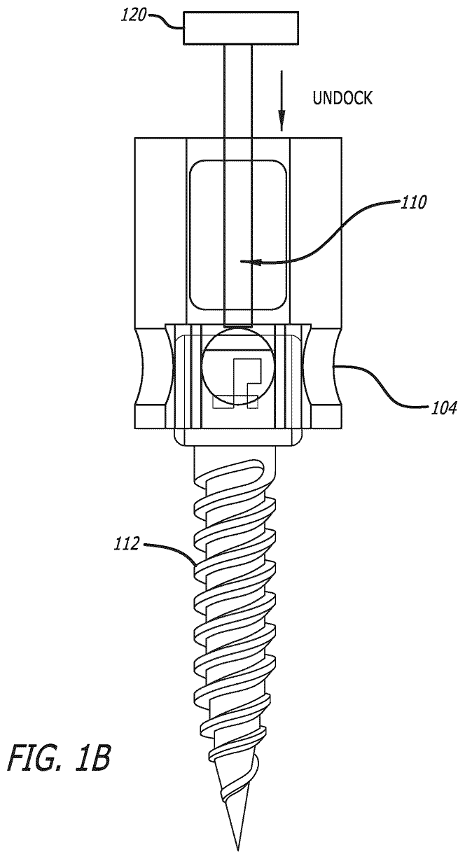

Certain embodiments will now be described in greater detail with reference to the figures. In FIG. 1A, depicted is a novel polyhedral fiducial marker (or bone marker, used interchangeably) configuration 100. The configuration 100 depicted in FIG. 1A includes an installation tool 102 and bone marker 112. The installation tool 100, which can also be referred to as a marker driver instrument assembly, which can be single procedure disposable or reusable, includes a driver shaft 104. The driver shaft 104 is configured as an elongated shaft. In some embodiments, as depicted in FIG. 1A, the proximal end of the driver shaft 104 can be configured with a screw or insertion means for which a medical professional (e.g., a surgeon) can utilize to driver the bone marker 112 into a patient. For example, as illustrated in FIG. 1A, the proximal end of the driver shaft 104 can include a handle 106 and a knob 108, which can be used to turn the tool as the bone marker 112 is deployed into a patient.

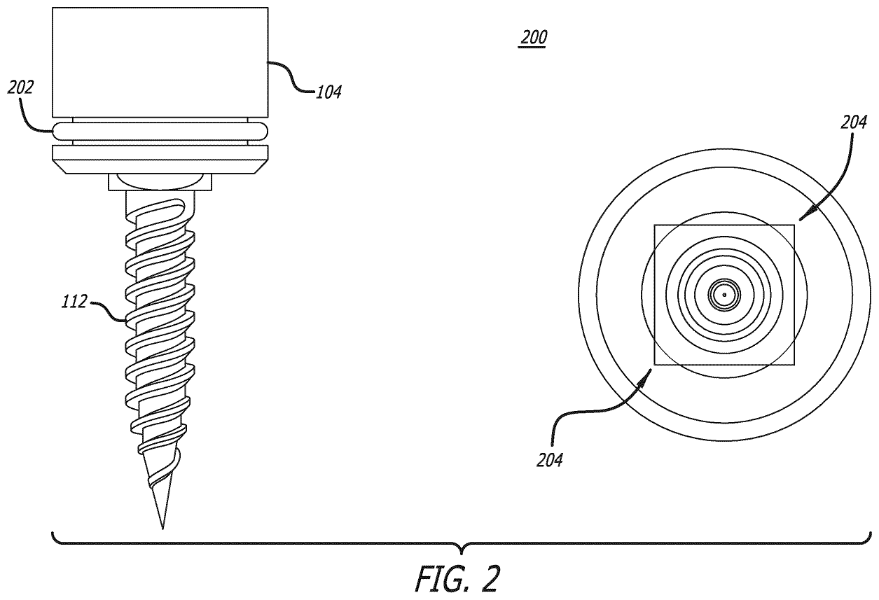

According to some embodiments, the distal end of the driver shaft 104 can include an opening or aperture. In some embodiments, the distal end of the driver shaft 104 can, therefore, be configured to accept, hold, and deploy the bone marker 112, and in some embodiments, as discussed in more detail below (at least in relation to FIGS. 2, 3A-3B and 6A-6C), include a component or instrument that can aide in bone marker retention and deployment into a patient.

In some embodiments, the distal end of the driver shaft 104 can be configured in a particular shape so as to accept the bone marker (or instrument/component). In some embodiments, the inner wall of the distal end of the driver shaft can be molded and/or shaped so as to fit the marker (or instrument/component). In some embodiments, the distal end of the driver shaft 104 can include a receptacle that defines an inside surface of the driver shaft 104, where the receptacle has a shape complimentary to an outside surface of a bone marker.

According to some embodiments, as illustrated in FIG. 1A, the distal end of the driver shaft 104 can include a rod magnet 110. According to some embodiments, the distal end of the driver 104 can be configured such that the inner wall of the driver shaft 104 has a shape complimentary to the outer walls and shape of the rod magnet 110. In some embodiments, the rod magnet 100 can have any type of known or to be known shape that can be used within an installation tool 102 to accept, hold and deploy a bone marker 112, such as, but not limited to, a cylinder, horseshoe, disc, sphere, ring, cube and the like. Accordingly, the distal end of the driver shaft 104 can be configured to fit the shaped rod magnet 110. For example, at least a portion of the inner surface of the distal end of the driver shaft 104 can be molded to fit a cylindrical shape when the rod magnet 110 is a cylindrical shape.

According to some embodiments, bone marker 112 can include a top portion and a bottom portion. The bottom portion of the bone marker can include a driver (or base screw) that attaches to the top portion (or head). As illustrated the driver is threaded for insertion into a patient. According to some embodiments, the top/head portion can be configured as a polyhedral shape. Such shape configuration provides novel installation means and additional surface (e.g., faces that provide increased surface area) for placement of fiducial markers, as discussed herein.

By way of background, as discussed above, conventional markers are cube shaped, and are configured to accept threaded rods from an installation tool for driving of the bone marker into a patient. At most, this therefore provides four (4) usable faces for placement of fiduciary markers. That is, the top and bottom surfaces are rendered unusable because they are subject to apertures for accepting and passing through the threaded rod of the installation tool. This, therefore, leaves only the remaining 4 exterior faces of the cube.

The disclosed bone marker 112 (and its implementation with the integrated rod magnet 110) enables additional faces to be utilized as fiduciary markers. That is, as illustrated in FIG. 1A, bone marker 112 has five (5) usable faces for placement of fiducial markers.

Moreover, the top face of marker 112 can be magnetized or coated with materials that contain magnetic properties, which enables retention by rod magnet 110 (e.g., held in position within tool 102 via the rod magnet 110). In some embodiments, the top surface's magnetic properties can be provided based on, but not limited to, the materials of the bone marker 112, materials of the fiducial markings, and/or other materials that are provided, coated, machined or injected into the marker 112).

According to some embodiments, marker 110 can be machined as a single component from 17-4 stainless steel (or an alternative ferrous metal). In some embodiments, the fiducial markings on each face of marker 110 can be provided via a two-step laser marking process that achieves fiducial markings (e.g., black and white) with a contrast that has values satisfying a threshold contrast (which enable detection via a camera system, for example).

By way of a non-limiting example, according to some embodiments, at the start of an ACL procedure, a bone marker 112 can be retained in the distal end of the driver shaft 104 via rod magnet 110, as depicted in FIG. 1A. Once the marker 112 is screwed and anchored into a patient (e.g., condylar bone of the patient's knee), the knob 108 and handle 106 on the proximal end of the driver shaft 104 can be rotated to release the marker 112.

Thus, according to some embodiments, installation tool 102 need not have the threaded rod disposed within the driver shaft 102 to hold and later release. Rather, as provided by the magnetic connectivity between the top surface of the marker 112 and the rod magnet 110 within the distal end of the driver shaft 104 of tool 102, once the fiducial marker 112 is placed within patient, the surgeon must simply pull the installation tool 102 away from the marker 112.

Turning to FIG. 1B, depicted is a non-limiting embodiment where configuration 100 (and tool 102) further includes a release button 120. In some embodiments, release button 120 can include a handle and a plunger, as illustrated in FIG. 1B. In some embodiments, the plunger of release button 120 can be configured as an elongated member that internally traverses the driver shaft 104 of tool 102. In some embodiments, the handle of the release button 120 can be accessible by an operating user of the tool 102 via an aperture on the elongated shaft, as discussed below.

According to some embodiments, the release button 120 can be configured as part of, or within the driver shaft 104, and configured for contact to the head of the marker 112. As discussed in more detail below, the release button 120 can be utilized (or in some embodiments, required) to operably release (or undock, used interchangeably) the bone marker 112 into the bone of a patient and thereby release the tool 102 from being engaged with the marker 112. As one of skill in the art would understand, the release button 120, and its engagement and undocking with the bone marker 112 after installation, aids in the reduction of loosening of the marker 112 in bone which is critical to the performance of tool 102/configuration 100.

According to some embodiments, the release button 120 can be operable to destabilize (and undock) from marker 112 when the delivery handle of the release button 120 radially turns (or rotates, or orbits, or moves) away from the axis of the marker a predetermined amount of degrees (and in some embodiments, in a predetermined direction). In some embodiments, such action can be caused via activating (or interacting with, e.g., pressing or depression) the release button, thereby causing the radial action of the plunger. Thus, for example, when the release button 120 is depressed and rotates a certain amount of degrees about the axis of the marker (e.g., 90 degrees in a direction), the tool 102 can be disconnected magnetically from marker 112.

In some embodiments, release button 120 can be configured so that user engagement can occur via a port, hole, aperture or other type of notch or opening that enables engagement and interaction with the release button 120. For example, the driver shaft 104 can be configured with an aperture on a side, or at the distal end, that enables the release button 120 to be engaged.

According to some embodiments, the release button 120 can be configured via or be coated with a magnetic material. In some embodiments, the release button 120 can be configured with a magnet residing at the distal end of the plunger of the release button 120. In some embodiments, a magnet can be rigidly coupled to the distal end of the release button 120 thereby enabling engagement with marker 112. According to some embodiments, the release button 120 can be retracted proximally (with a predetermined amount of force and/or duration of time) to break the magnetic retention with marker 112.

In FIG. 2, depicted is a non-limiting embodiment of a polyhedral fiducial marker configuration 200 according to some embodiments of the present disclosure. According to some embodiments, configuration 200 can be configured with a retention member to hold and release marker 112 (which should not be limiting, in that from the discussion below, additional and/or alternative forms of markers can be utilized via configuration 200 without departing from the scope of the instant disclosure).

Configuration 200, at the distal end of driver shaft 104 of installation tool 102, includes the retention member 202. According to some embodiments, the retention member can be comprised of an elastomeric member and molded into a ring shape. Thus, in some embodiments, member 202 can be an O-ring 202. O-ring 202 can be circular in shape, and molded/configured so as to conform to an external shape of installation tool 102, as illustrated in FIG. 2.

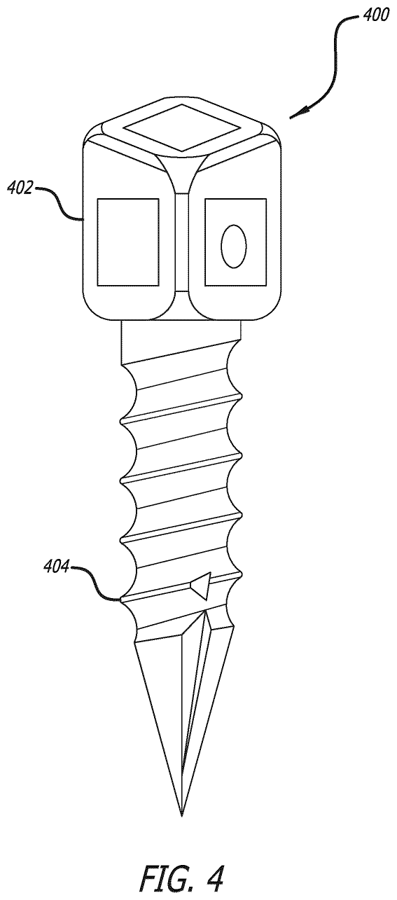

According to some embodiments, O-ring 202 can be utilized as an alternative to the rod magnet 110 (from FIG. 4). In some embodiments, the O-ring 202 can be sized and situated/positioned within an O-ring groove within the distal end of the driver shaft 104 in order to provide a friction fit on at least two corners of the marker 112, thereby affording a reliable retention force on the marker 112 during placement and retrieval. A non-limiting example of the friction fit related to the corners of marker 112 is depicted in FIG. 2 via items 204. Thus, for example, bone marker 112 is inserted into tool 102 and held in position via items 204 clipping over the lower corners of head of marker 112. According to some embodiments, O-ring 202 can be configured to interact with notches and/or grooves on the corners of the marker 112; however, such interaction does not interfere with the fiducial patterns on the faces of the marker 112.

Thus, according to some embodiments, installation tool 102 need not have the threaded rod disposed within the driver shaft 104 to hold and later release. Rather, as provided by the O-ring 202 functionality, once the fiducial marker 112 is placed within patient, the surgeon must simply pull the installation tool 102 away from the marker 112. In some embodiments, a release button 120 can be utilized to destabilize the marker 112 from the tool 102, as illustrated in FIG. 1B, and discussed above.

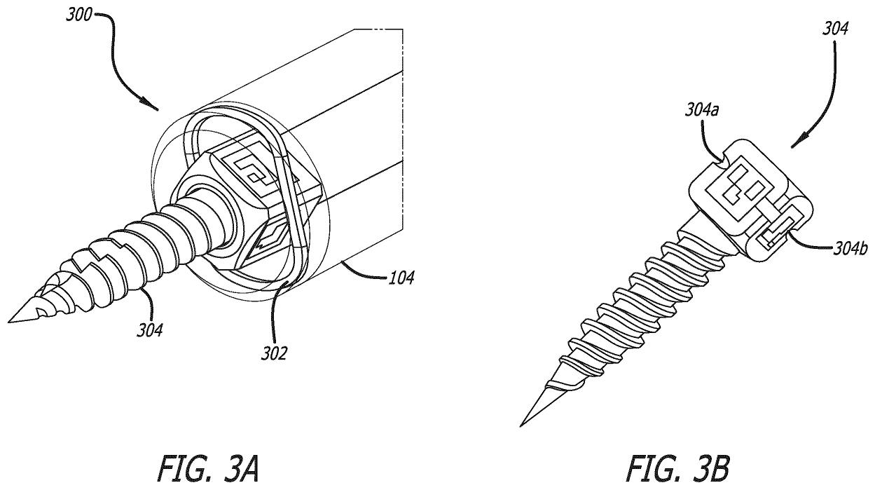

In FIGS. 3A-3B, depicted are non-limiting embodiments of a polyhedral fiducial marker configuration 300 and a utilized bone marker 304 according to some embodiments of the present disclosure. Configuration 300 can be utilized as an alternative to the O-ring configuration 200 discussed above in relation to FIG. 2.

Turning to FIG. 3A, configuration 300 includes a retention member configured as a spring clip 602 (or spring clip mechanism, used interchangeably). Spring clip 302, which is comprised of an elastomeric material and positioned at the distal end of the driver shaft 104 an installation tool 102, is a clip retainer that engages a corresponding groove(s) or notch(es) machined into the edges of a five-sided marker (e.g., marker 304 of FIG. 3B, as discussed herein). Spring clip 302 can be configured with geometry to fit within the inside wall of the distal end of the driver shaft 104 of an installation tool. In some embodiments, the shape of the spring clip 302 can be any shape that enables retention with a threshold satisfying amount of friction to hold and release a bone marker. In some embodiments, such shape can be, but is not limited to, a rectangle (e.g., as illustrated in FIG. 3A), a square, circular, oval, and the like, or some combination thereof.

In some embodiments, a release button 120 can be utilized to destabilize the marker 112 from the tool 102, as illustrated in FIG. 1B, and discussed above.

Turning to FIG. 3B, bone marker 304 is depicted. As discussed above, marker 304 can include grooves or notches, as identified via items 304a and 304b. It should be understood that while only two grooves/notches are depicted in FIG. 3B, it should not be construed as limiting, as additional grooves/notches can be included on additional or the same edges of the head of the bone marker 304 without departing from the scope of the instant disclosure. According to some embodiments, the placement, positioning, size (e.g., depth) and quantity of groove/notches (e.g., 304a, 304b) on marker 304 can directly correspond to a type and configuration of spring clip 302, which enables a secure but releasable retention of marker 304 during a medical procedure.

According to some embodiments, as the marker 304 is inserted into the driver shaft 104 of installation tool 102, the spring clip 302 expands until it snaps into an edge groove (304a, 304b) of the maker 304 to lock the marker within the driver shaft 104. Thus, according to some embodiments, via functionality provided by the spring clip 602, once the bone marker 304 is placed within the patient, the surgeon must simply pull the installation tool 102 away from marker 304 and spring clip 302 will release the marker 304, the surgeon must simply pull the installation tool 10 away from the marker 402.

Turning to FIG. 4, depicted is a non-limiting example embodiment of polyhedral fiducial marker 400 according to some embodiments of the present disclosure.

In FIG. 4, marker 400 is depicted. Marker 400 has a cube shape 402 that is configured as a five (5) surface marker with a slanted top face. The slanting of the top face, which is slanted at an angle within a predetermined range so as to enable detection while securing the driver 404 to the cube 402, enables an additional top surface from existing cube configurations. According to some embodiments, the slanted top face of cube 402 provides increased visibility from an arthroscope and camera system, thereby increasing marker detectability and accuracy under operational conditions (e.g., fluid, tissues, debris, and the like).

In some embodiments, marker 400 can be secured to a driver through a “spring loaded ball plunger” mechanism that can actuate on one of the side faces of cube 402. The side face fiducial pattern of cube 402 can either be inexistent (by using a cube face that usually faces away from the camera viewpoint) or have a design that would allow the inclusion of the spring ball inset without deteriorating the detectability and accuracy of such face.

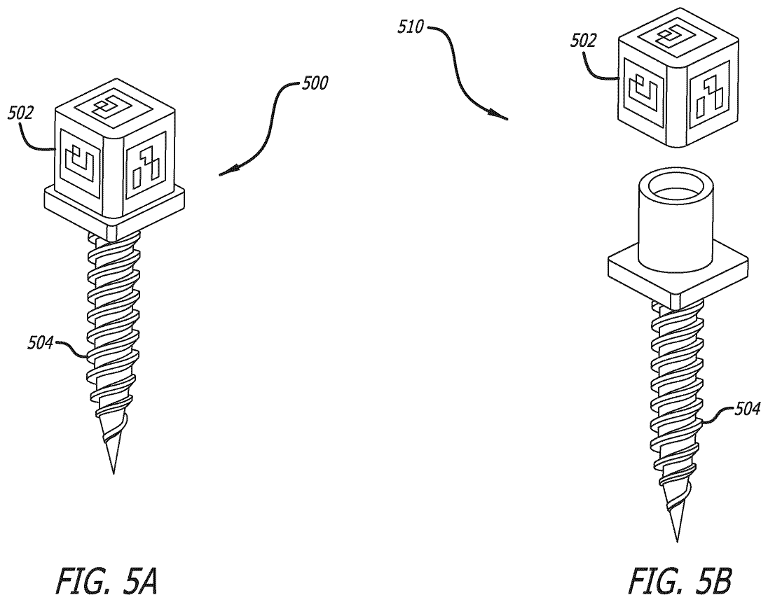

Turning to FIGS. 5A-5B, depicted are non-limiting example embodiments 500 and 510 of a polyhedral fiducial marker according to some embodiments of the present disclosure. Embodiments 500 and 510 provide non-limiting example modifications of bone marker 112, as discussed above in relation to FIG. 1A, and discussed below.

Turning to FIG. 5A, according to some embodiments, marker 500 can be manufactured using an injection molded thermoplastic polymer (e.g., Polyether ether, PEEK or Polyetherimide PEI, for example). In some embodiments, marker 500, which is a five-sided design, can be a two-piece assembly, where the cube portion 502 of the marker 500 can be attachable and detachable to the driver 504 (or base screw) of the marker 500. FIG. 5A depicts an embodiment where the portions are attached, and FIG. 5B depicts an embodiment illustrating the detachment of the portions.

According to some embodiments, the cube portion 802 can be attached and/or affixed to the driver 804 according to any known means for securely and temporarily attaching two surgical pieces, such as, but not limited to, by screwing, via magnets, via an adhesive, and the like, or some combination thereof.

In some embodiments, the markers of embodiments 500 and 510 can be machined from 17-4 stainless steel (or an alternative ferrous metal, as discussed above) for retention within an installation tool via a rod magnet (in a similar manner as discussed above in relation to FIGS. 1A and 1B).

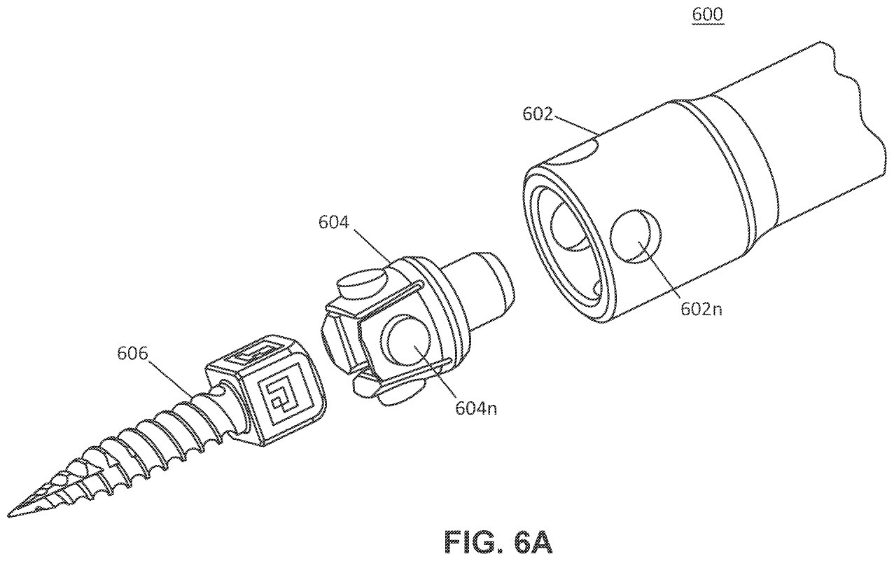

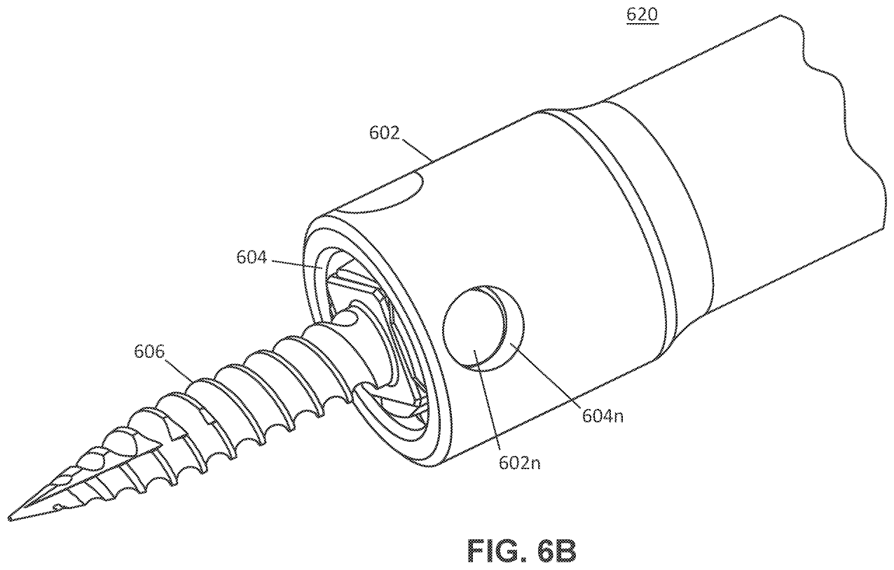

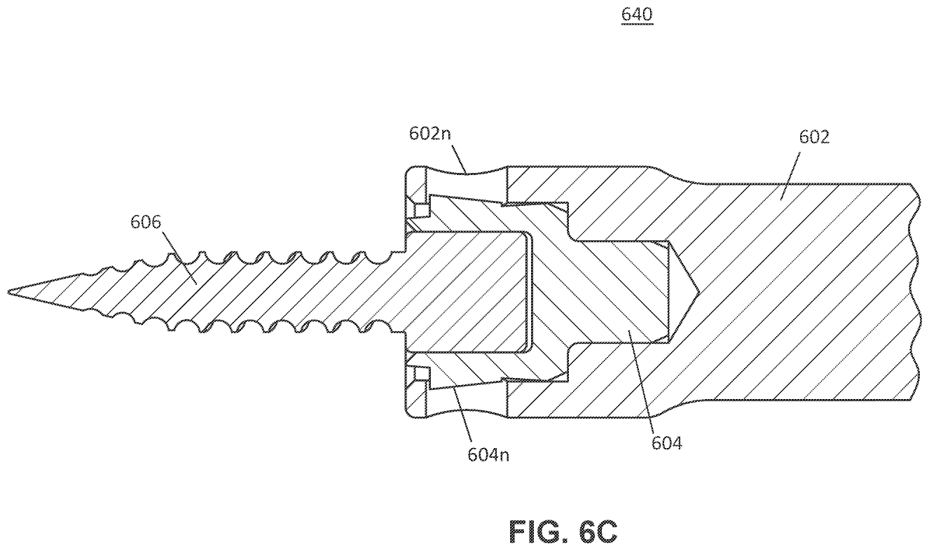

Turning to FIGS. 6A-6C, depicted are non-limiting example embodiments of a polyhedral fiducial marker configurations 600, 620 and 640 according to some embodiments of the present disclosure.

As depicted in FIG. 6A, according to some embodiments, configuration 600 includes an installation tool 602, flexible plastic component 604 and bone marker 606. In some embodiments, installation tool 602 can be configured in a similar manner as installation tool 102 as discussed above. In some embodiments, installation tool 602 can have its distal end configured for acceptance and retention of component 604. Accordingly, as discussed below, tool 602 can include a set or plurality of apertures 602n (or notches or grooves, and the like), where the number, shape and size of each aperture 602n directly corresponds to the number, shape and size of protrusions (or “spring tabs”) 604n on component 604.

In some embodiments, marker 606 can be any type of marker discussed herein, such as, for example, marker 112 (e.g., a 5-sided marker).

In some embodiments, the flexible plastic component 604 (also referred to as a friction retainer or retention member, user interchangeably) can be configured to be positioned within and fit within the distal end (or a receptacle of the distal end) of the installation tool 602.

As illustrated in FIG. 6A, in some embodiments, the flexible plastic component 604 can include an inside surface configured about an outside surface of a bone marker 606, and an outside surface that defines a protrusion(s), which is configured to interact with an aperture 602n through a side wall of component 604.

As mentioned above, in some embodiments, the flexible plastic component 604 can be configured with a predetermined number of protrusions (e.g., referred to as “spring tabs”) 604n. In some embodiments, each spring tab 604n can be configured to fit within a corresponding number of apertures 602n within the distal end of the installation tool 602. This enables the tool 602 to accept and hold component 604 and subsequently marker 606, as discussed herein. In some embodiments, as discussed below, the spring tabs 604n of the flexible plastic component 604 can expand when the maker 606 is inserted into component 604, as discussed below.

In some embodiments, as illustrated in FIG. 6B, configuration 620 is a depicted view of configuration 600 where the flexible plastic component 604 is pressed into the distal end of the driver shaft of the installation tool 602. As a result, the marker 606 has been inserted into the flexible plastic component 604 such that the spring tabs 604n have expanded accordingly to engage with the apertures 602n. In some embodiments, retention force maintaining the marker 606 within component 604 (and tool 602) is the friction force between the flexible plastic component 604 and the marker 606.

According to some embodiments, when the spring tabs 604n of the flexible plastic component 604 are expanded, they have line-to-line to near line-to-line contact with the inside of the distal tip of the installation tool 602. Accordingly, this allows torque generated during insertion of marker 606 into bone to be transferred to the driver of the marker 606.

According to some embodiments, the spring tabs 604n of the flexible plastic component 604 can also have features on their exterior that engage with apertures 602n on the driver shaft of the installation tool 602 to keep the friction retainer from separating from the driver shaft. According to some embodiments, such features can be based on the materials of the spring tabs 604n and/or other injected or coated materials provided to the surface of the spring tabs 604n and/or apertures 602n.

According to some embodiments, to remove the marker 606 from the flexible plastic component 604, the surgeon only needs to axially pull on a handle (at the proximal end, as discussed above in relation to at least FIG. 1A) of the installation tool 606, in a similar manner as discussed above.

According to some embodiments, the flexible plastic component 604 can be made from and/or comprise of material that is sufficiently soft (e.g., a material with a softness characteristic(s) that satisfies a softness threshold) so that it does not affect etched fiducial markings (e.g., quick response (QR) codes) on the marker 606. In some embodiments, the spring tabs 604n of the flexible plastic component 604 can have relief cuts to prevent them from contacting fiducial markings (e.g., QR codes) on the marker 606. Alternatively, in some embodiments, instead of engaging the flats of the head of the marker 606, the spring tabs 604n of the flexible plastic component 604 can engage the at least two corners of the head of the marker 606 to prevent contact with the fiducial markings (e.g., QR codes), which can be performed in a similar manner as discussed above in relation to FIG. 2.

FIG. 6C illustrates configuration 640, which is a schematic depiction of configuration 620. Accordingly, configuration 640 depicts bone marker 606 being engaged within component 604, which itself is engaged within the distal end of installation tool 602, whereby spring tabs 604n have engaged with apertures 602n.

According to some embodiments, the flexible plastic component 604 can be designed to only undergo elastic deformation so that it may reengage the marker 606, if necessary.

In some embodiments, configurations 600, 620 and 640 can be further modified to include the release button 120 discussed above in relation to FIG. 1B, which can enable a secure stabilization of a marker and release into a patient, as discussed above.

Accordingly, in some embodiments, by utilizing the flexible plastic component 604 to engage the marker 606, configurations 600, 620 and 640 enable the use of markers with additional faces (e.g., marker 606, as discussed above), which enables an improved visualization of the marker's QR codes during a procedure.

Those skilled in the art will recognize that the methods and systems of the present disclosure may be implemented in many manners and as such are not to be limited by the foregoing exemplary embodiments and examples. In this regard, any number of the features of the different embodiments described herein may be combined into single or multiple embodiments, and alternate embodiments having fewer than, or more than, all of the features described herein are possible. Indeed, functionality may also be, in whole or in part, distributed among multiple components, in manners now known or to become known.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

1. A method of installing a bone marker, the method comprising:
retaining at least the bone marker at a distal end of an installation tool, the installation tool defining a longitudinal central axis and a handle on a proximal end;
installing the bone marker into a bone by turning the installation tool about the longitudinal central axis; and
disconnecting the installation tool from the bone marker and leaving the bone marker in the bone by pulling the installation tool away from the bone without interacting with a mechanical release on the handle.

2. The method of claim 1, wherein the installation tool comprises an elastomeric material at the distal end of the installation tool, wherein the retaining of at least the bone marker is enacted via the elastomeric material.

3. The method of claim 2, wherein the elastomeric material comprises at least one of an O-ring and spring clip.

4. The method of claim 1, wherein the installation tool comprises a flexible component that is inserted within the distal end of the installation tool, the flexible component configured with a set of spring tabs that engage in at least near line-to-line contact with the distal end of the installation tool.

5. The method of claim 4, wherein the flexible component accepts the bone marker, and based on the accepted bone marker, the set of spring tabs expand for the engagement within the distal end of the installation tool.

6. The method of claim 4, wherein the expanded spring tabs of the flexible component in the distal end of the installation tool comprises a retention force for maintaining the marker within flexible component, the retention force being a friction force between the flexible component and the bone marker.

7. The method of claim 1, wherein the bone marker is a polyhedral fiducial marker, the bone marker comprises a head and a driver, wherein the head comprises a geometric shape with a predetermined number of faces, wherein each face comprises a fiducial marker detectable by a camera system, wherein the driver is threaded for insertion into a bone of a patient.

8. The method of claim 7, wherein the head of the bone marker is at least one selected from a group comprising; a hexahedron shape, wherein the head comprises at least four surfaces for placement of the fiducial marker; a hexahedron shape, wherein the head comprises at least five surfaces for placement of the fiducial marker; and a slanted top surface, the top surface being usable for placement of a fiducial marker.

9.-10. (canceled)

11. The method of claim 7, wherein each face comprises at least one selected from a group comprising: a fiducial pattern with a same pattern; and a fiducial pattern with unique pattern.

12. (canceled)

13. An installation tool for a bone marker, the installation tool comprising:
an elongated shaft defining a distal end and a proximal end;
a handle disposed at the proximal end of the elongated shaft;
a receptacle disposed at the distal end, the receptacle defines an inside surface having a shape complementary to an outside surface of a bone marker, the bone marker comprising a set of fiducial markers; and
a means for retaining and releasing the bone marker from the receptacle, the means for retaining and releasing operable without interaction by a user interacting with the handle.

14. The installation tool of claim 13, wherein the means for retaining and releasing further comprises a magnet disposed within the elongated shaft proximal to the receptacle.

15. The installation tool of claim 13, wherein the means for retaining and releasing further comprises a retention member disposed in operational relationship to the receptacle, the retention member configured to interact with a notch or groove of the bone marker to retain bone marker within the receptacle.

16. The installation tool of claim 15, wherein the retention member is at least one selected from a group consisting of: an elastomeric material; an O-ring; and a spring.

17. The installation tool of claim 13, wherein the means for retaining and releasing further comprises a friction member configured to be disposed between the bone marker and the inside surface of the receptacle, the friction member including:
an inside surface configured to abut an outside surface of the bone marker; and
an outside surface defining a protrusion, the protrusion configured to interact with an aperture through a side wall of the receptacle.

18. The installation tool of claim 17, wherein friction member holds the bone marker within the receptacle by retention force, and where the installation tool is configured to release the bone marker by a force applied along the longitudinal central axis overcoming the friction force.

19. The installation tool of claim 18, wherein the retention force is based on a friction force between the retention member and the bone marker.

20. The installation tool of claim 13, wherein the retention member is configured with relief cuts in order to prevent contact with the set of fiducial markers.

21. A method of installing a bone marker, the method comprising:
retaining at least the bone marker at a distal end of an installation tool, the installation tool defining a longitudinal central axis and a handle on a proximal end;
installing the bone marker into a bone by turning the installation tool about the longitudinal central axis; and
disconnecting the installation tool from the bone marker and leaving the bone marker in the bone by pulling the installation tool away from the bone.

22. The method of claim 21, wherein the installation tool further comprises a release button configured to contact the bone marker.

23. The method of claim 22, further comprising:
upon the installation of the bone marker into the bone, activating the release button to distally control the disconnection of the installation tool from the bone marker.

24. An installation tool for a bone marker, the installation tool comprising:
an elongated shaft defining a distal end and a proximal end;
a handle disposed at the proximal end of the elongated shaft;
a receptacle disposed at the distal end, the receptacle defines an inside surface having a shape complementary to an outside surface of a bone marker, the bone marker comprising a set of fiducial markers; and
a release button comprising a plunger, the plunger configured as an elongated member that internally traverses the elongated shaft, the plunger accessible by an operating user of the installation tool via an aperture on the elongated shaft.

25. The installation tool of claim 24, wherein upon the installation of the bone marker into a bone, activating the release button to distally disconnect the installation tool from the bone marker.
